(19) United States
(12) Patent Application Publication
(10) Pub. No.: US 2012/0323083 A1
(43) Pub. Date: Dec. 20, 2012

(54) Lighting System

(76) Inventor: Graeme Hall, Yorkshire (GB)

(21) Appl. No.: 13/581,772

(22) PCT Filed: Mar. 2, 2011
(86) PCT No.: PCT/GB11/50417

§ 371 Date: Aug. 29, 2012

(30) Foreign Application Priority Data
Mar. 18, 2010 (GB) 1004490.7
Nov. 1, 2011 (GB) 1018415.8

(51) Int. Cl. A61B 1/06

(52) U.S. Cl. 600/249

(57) ABSTRACT

A lighting system for medical use which comprises a light assembly and a drape. The light assembly comprises an elongate arm which is surface mountable from a proximal end of the arm, a light head connected to a distal end of the arm and a light source mounted in the light head. The drape comprises an elongate flexible body and a substantially transparent portion. The drape is mounted or mountable on and substantially encloses the light assembly such that in use an amount of the light from the light source exits the substantially transparent portion.

The present invention relates to a lighting system.

In medical environments, patient interactions are often compromised due to lack of good lighting and the need for the clinician to protect the patient from infections. Medical applications are particularly difficult to illuminate effectively and medical staff have to protect themselves and patients from cross infection by wearing sterile gloves. Indeed the risk of hospital acquired infections transferred from items in the healthcare environment such as lighting equipment, furniture and white coats is now well known and the subject of great concern and publicity.

When medical staff wish to closely examine a patient in the office or conduct a minor procedure such as remove sutures or aspirate a seroma, they may use an examination light which may be a wall mounted light, a mobile light on a heavy stand or a pen-light. Most examination lights do not have sterilisable handles and present a risk of cross infection. In particular, gynaecological examination and routine smear tests are commonly carried out using examination lights without sterilisable handles. Cervical cancer is now known to be spread by infection by certain strains of the human papilloma virus and this practice creates a substantial risk of infections being spread from patient to patient.

During precise work in a confined area, a surgeon in an operating theatre is hampered by large, heavy, difficult to control ceiling mounted lights that are out of the sterile field. The lights bathe the surgeon in heat and often lead to minor injuries from collision with the surgeon. Collisions between the surgeon's head and the light and incidences of the surgeon inadvertently touching the non-sterile parts of the operating lamp with his gloved hands are behind incidences of contamination with a corresponding risk of infection to the patient.

Incidents of infection in orthopaedic surgery are particularly severe as infections in the bones are difficult to treat with antibiotics and can often lead to further surgery and amputations. Head lights are disliked by most surgeons as they demand the operator look at the area where the light is focused whereas the situation often demands a surgeon look from another perspective.

Known operating theatre lights have a removable handle cover which can be sterilized using an autoclave and replaced for each operation. An alternative method deploys single use, sterile handle covers which slide into place over a handle on the operating theatre light and are disposed of after each operation. In use, these handle covers fit tightly to the operating light handle.

Most known examination lights have no provision for sterile handling. Those that do offer similar provision to known operating theatre lights

The present invention seeks to improve lighting systems for medical use by deploying a physical barrier which can be sterilised or replaced between uses but which does not compromise the operation of the light assembly. This reduces the risk of infection.

Thus viewed from one aspect the present invention provides a lighting system for medical use comprising:

a light assembly which comprises:
an elongate arm which is surface mountable from a proximal end of the arm,
a light head connected to a distal end of the arm, and
a light source mounted in the light head; and
   a drape which comprises:
an elongate flexible body, and
a substantially transparent portion,
   wherein the drape is mounted or mountable on and substantially encloses the light assembly such that in use an amount of the light from the light source exits the substantially transparent portion.

In the lighting system of the invention, the drape advantageously provides a physical barrier which reduces the risk of infection. The drape may also reduce the risk of contamination of the light assembly itself.

The lighting system may be sterilised between uses by replacing the used drape with a sterile drape or by sterilising the used drape.

The lighting system is suitable for medical use. The lighting system may be composed of materials which are approved for medical use. The parts or materials of the lighting system may satisfy relevant accepted standards for medical equipment.

Preferably the light source is a medical light source. A medical light source may provide light of a suitable intensity and colour for medical use. A medical light source may comply with one or more of the International Standards IEC60601, IEC60601-1 and IEC60601-2-41.

Heat and light generated by the light source originate at the distal end of the arm (i.e. remote from the proximal end of the arm).

Preferably the light source is a low heat emittance light source. For example, the light source in use preferably emits a sufficiently small amount of heat that the drape or the light source do not overheat.

The light source may be a powered light source. Preferably the light source has a power consumption of less than 50 W. The light source may have a power consumption of less than 40 W. The light source may have a power consumption of less than 30 W. The light source may have a power consumption of less than 20 W. The light source may have a power consumption of less than 10 W. The light source may have a power consumption of less than 5 W. The light source may have a power consumption of at least 1 W. The light source may have a power consumption of at least 5 W. The light source may have a power consumption of at least 10 W. The light source may have a power consumption of at least 20 W. The light source may have a power consumption of at least 30 W. The light source may have a power consumption of at least 40 W.

The light source may be a solid state light source. Preferably the light source comprises an LED (light emitting diode) or HD-LED. The light source may comprise a semiconductor, organic or polymer LED.

The light source may comprise a plurality of LEDs. The light source may comprise a combination of different coloured LEDs (eg Red Green Blue or Red Green Blue Orange).

LEDs do not emit a significant amount of infra red radiation as part of the light emission spectrum. This reduces the amount of heat produced by an LED light source. In addition, LED light sources have much higher efficiencies than tungsten halogen light sources. The efficiency of high performance LED light sources is typically 5 to 10 times higher than tungsten halogen which makes them suitable light sources for this invention by virtue of lower heat generation than tungsten halogen light sources.

The light source may comprise a fluorescent or gas discharge light source. Fluorescent and gas discharge light sources also meet the requirements for a low heat emittance light source for use inside a drape.

The light source may have a high colour rendition. The light source may have a colour temperature in the range of natural daylight. The light source may have a high colour rendition in the visible red wavelengths of the spectrum. The light source may have a colour rendition index with Ra>90 and/or R9>90.

The light quality from the light source may be equivalent to that of an operating theatre light or a medical examination light.

The light source may have adjustable intensity. The light source may have an adjustable beam size or field size. The light source may have adjustable light colour (for example, adjustable colour rendition, colour temperature and/or red balance).

The light assembly is surface mountable via the proximal end of the elongate arm. The light assembly may be mountable to medical equipment. The light assembly may be mountable to an operating theatre table. The light assembly may be mountable to a medical rail. The light assembly may be pole mountable, wall mountable, gurney mountable, bed rail mountable, pendant mountable, mountable from a mobile base or ceiling mountable.

Preferably the light assembly is not ceiling mounted.

The light assembly may be fixed. Preferably the light assembly is portable. The light assembly may be free standing. For example, the light assembly may be self-supporting when positioned on a surface without physical attachment to the surface.

The elongate arm may be moveable (eg pivotable, bendable or rotatable). The elongate arm may comprise a first end and a second end. The first end may be a proximal end and the second end may be a distal end. The second end may connect directly or indirectly to the light head. The second end may comprise a light head connector.

The elongate arm may comprise a flexible portion. The elongate arm may comprise a rigid portion. The elongate arm may comprise a plurality of rigid portions or flexible portions or a combination of one or more rigid portions and one or more flexible portions.

The light assembly may further comprise a mounting point. The mounting point may be directly or indirectly connected to the first (or proximal) end of the elongate arm. The mounting point may allow the arm to be mounted on one or more of medical equipment, an operating theatre table, a medical rail, a pole, a wall, a gurney, a bed, a pendant, a mobile base or a ceiling.

The light assembly may further comprise a base. The base may be attached to the elongate arm through the mounting point.

The light head connector may allow the light head to be moveable or fixed with respect to the elongate arm. The light head connector may allow the light head to pivot and/or rotate on the elongate arm.

The light assembly may further comprise one or more light controls. The light controls may be located on the elongate arm or the light head. The light controls may allow the light source to be switched on and off and control the intensity and/or colour of the light source. Alternatively, the light assembly may be operated by remote control.

The light assembly may be partly or fully enclosed by the drape. For example, the drape may enclose solely the light head and the elongate arm. The mounting point may be non-enclosed by the drape (i.e. substantially free of the drape).

An operator may be able to handle the light assembly without loss of sterility due to the drape. The drape may allow the operator to position the light head and operate the light controls without loss of sterility.

The drape may be suitable for single use. The drape may be suitable for multiple uses. Preferably the drape is single use. Preferably the drape is disposable.

The drape may be a sterile drape. The drape may be pre-sterilised. The drape may be sterilisable. For example, the drape may be sterilisable in an autoclave.

The drape may comprise an opening. The elongate flexible body may comprise an open end. Preferably the elongate flexible body is open-ended. The elongate flexible body may be a sleeve. Preferably the elongate flexible body is a sock.

Preferably the substantially transparent portion is located at or near to an end (for example the distal end) of the elongate flexible body. The substantially transparent portion may be located at or near to an end opposed to the open end. Particularly preferably the substantially transparent portion is located at the foot of the sock.

The drape may be collapsible (for example radially or longitudinally collapsible). The drape may be concertinable. The drape may be corrugated (eg fluted) or non-corrugated.

The elongate flexible body may grip the light assembly at one or more gripping points. Alternatively the elongate flexible body may be substantially free of the light assembly.

The drape may be tetherable or tethered to the light assembly at one or more tethering points.

The drape may be attachable or attached to the light assembly at one or more attachment points. The drape may be attachable to the elongate arm. The drape may be attachable to the mounting point. The drape may be attachable to the light head. The drape may comprise an attachment aid to aid attachment to the light assembly.

The drape may form a seal against the elongate arm. The seal may be near to the mounting point. The seal may be formed by an elastic waist in the drape. Alternatively the seal may be formed by an adhesive section on the drape.

The drape may be retainable or retained on the light assembly at one or more retaining points. The drape may comprise one or more ties (eg elastic ties) to retain the drape at the one or more retaining points. A tie may be used to constrict the drape around the elongate arm.

The drape may fully enclose (eg encapsulate) the light assembly. The drape may encapsulate the light assembly by sealing an opening in the drape.

The drape may partially enclose the light assembly. Preferably the drape encloses the light assembly short of the mounting point. This advantageously allows the operator to touch the light head, light controls and the elongate arm as far as the mounting point. The drape may have an opening to allow the mounting point to engage the surface on which it is mounted.

The drape may be composed of a sterilisable material. The drape may be composed of a material which is approved for medical use.

The drape may be composed of a first polymeric material. Examples of suitable polymeric materials include thermoplastic polymers such as polyolefins, polycarbonates, polyamides and polyesters. The first polymeric material may be flexible. The first polymeric material may be an elastomer. The first polymeric material may be optically clear.

The elongate flexible body may be composed of the first polymeric material. Preferably the elongate flexible body is composed of a polyolefin. For example the elongate flexible body may be composed of polypropylene.

The substantially transparent portion of the drape may have substantially the same optical transmittance as the flexible body. Preferably the substantially transparent portion has a higher optical transmittance than the flexible body.

The substantially transparent portion may be composed of the first polymeric material.

Alternatively the substantially transparent portion may be composed of a second polymeric material. Examples of suitable polymeric materials include thermoplastic polymers such as polyolefins, polycarbonates, acrylics, polyamides and polyesters and thermosetting polymers such as curable resins (for example epoxies, poly aryl ketones). The second polymeric material may be optically clear. The second polymeric material may be rigid. The second polymeric material may have a higher melting point than the first polymeric material.

The substantially transparent portion may be optically clear. The substantially transparent portion may be rigid. The substantially transparent portion may be a window.

Preferably the substantially transparent portion is made from polycarbonate, acrylic or polyester. The polyester may be PET. The polyester may be polyethylene terepthalate glycol.

The elongate flexible body and substantially transparent portion may be joined by ultrasonic welding, heat sealing, solvent welding or by adhesives.

The lighting system may be used as an auxiliary surgical light in operating theatres. The lighting system may be small relative to the main operating theatre light. The lighting system may be used for minor procedures and/or as an examination light outside the operating theatre.

For example the lighting system may be used in one or more of the following medical procedures: gynaecological examination, smear tests, orthopaedic surgery, perineal surgery, general surgery, colorectal surgery, gynaecological operations, laparoscopic surgery, head and neck procedures, ear-nose-throat procedures and paediatric procedures.

The lighting system may be used in speciality medical units such as Intensive Care Units, Critical Care Units and post operative recovery areas.

The lighting system may be used in an ambulance or emergency vehicle. In an ambulance, paramedics may used the lighting system in place of a flashlight or the ambient light in the vehicle.

The lighting system may be used in an Accident & Emergency department in place of general overhead lighting. It is not unusual for a trauma victim to need multiple procedures to be performed contemporaneously by several healthcare professionals—urinary catheterization, intubation, central venous catheterization, chest drainage and peritoneal lavage. Multiple lighting systems may be used in these cases. While most Accident & Emergency Departments have trauma bays, these have limited feature lighting and the remaining cubicles often just have regular overhead room lights.

The lighting system may assist in medical procedures where there is a need for medical light and a risk of infection or cross contamination to be carried out without risking loss of sterility from direct contact with the light assembly.

A major area in which the lighting system may be used is women's health and gynaecology. The use of a sterile drape will allow a Clinician to carry our gynaecological examinations, smear tests and procedures with good lighting, that can be moved into position without risk of contamination from touching the light assembly directly.

The lighting system may be used in hospital consulting rooms, primary care (family doctors, general practitioners and clinics), accident and emergency and ambulances where non-sterile examination lights are currently being used with a consequential risk of infection.

Viewed from a further aspect the present invention provides a kit of parts comprising a light assembly and a drape mountable on the light assembly as hereinbefore defined.

Viewed from a yet further aspect the present invention provides a drape as hereinbefore defined.

An embodiment of the present invention will now be described by way of example only with reference to the accompanying drawings in which:

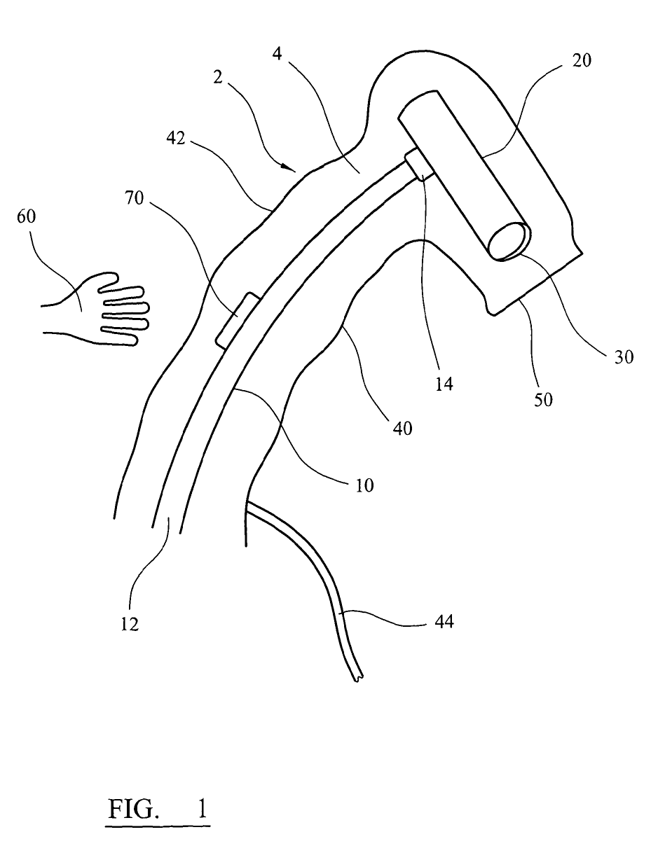

FIG. 1 is a schematic view of an embodiment of the present invention;

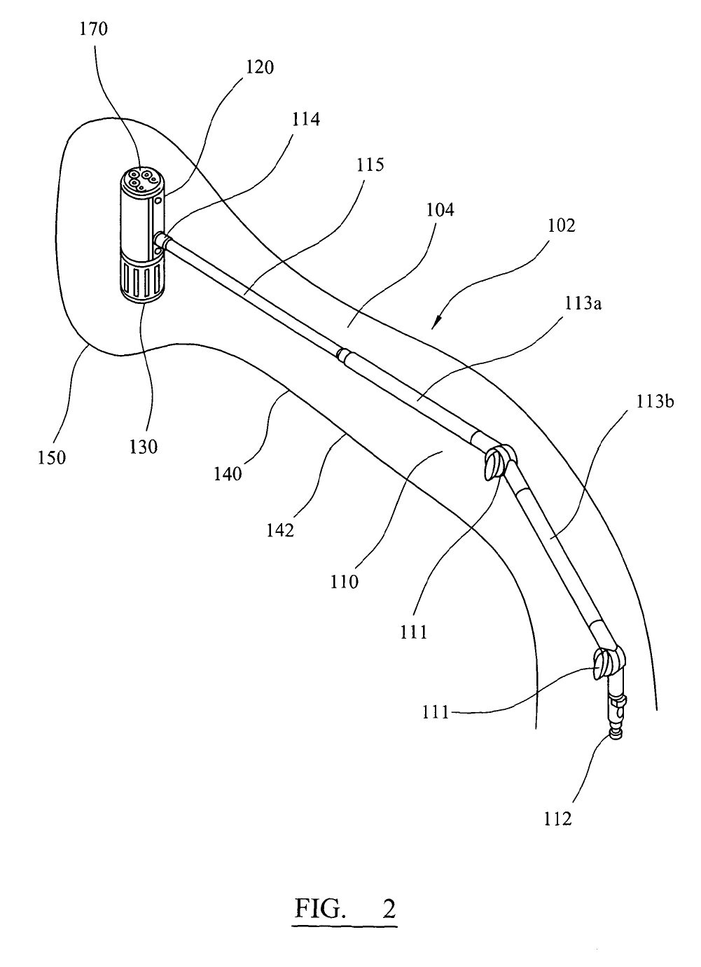

FIG. 2 is a view of a second embodiment of the present invention; and

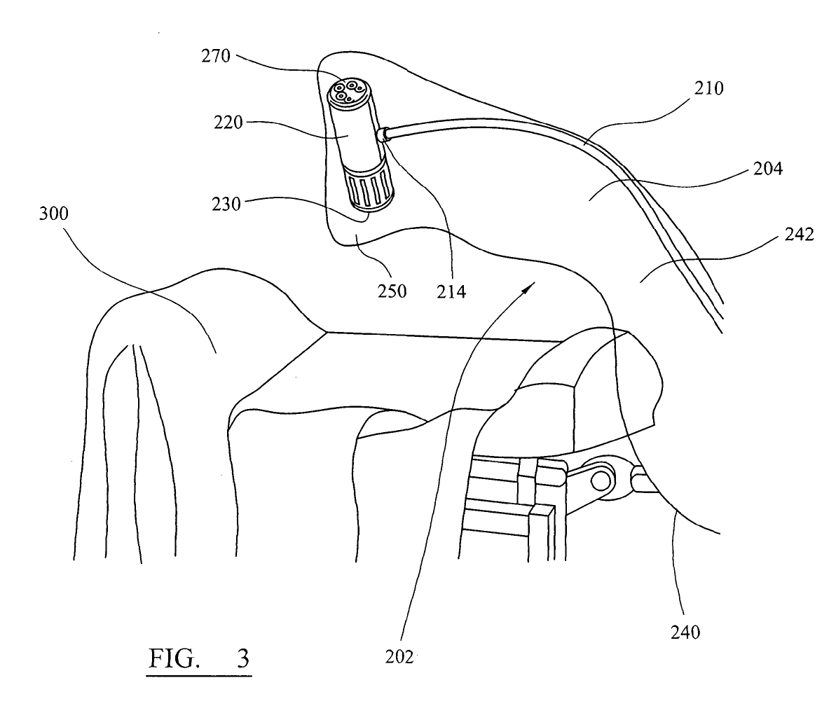

FIG. 3 is a view of a third embodiment of the present invention.

FIG. 1 shows a lighting system 2 which comprises generally a light assembly 4 and a sterile drape 40.

The light assembly 4 comprises a semi-rigid, infinitely moveable arm 10 which is (for example) mountable to a stand via a mounting point 12 at a first (proximal) end of the aim 10. The second (distal) end of the arm 10 is connected to a light head 20 by a light head connector 14. The light head connector 14 allows the light head 20 to rotate and pivot relative to the arm 10.

An LED light source 30 with a power consumption of less that 50 W is mounted in the light head 20.

Light controls 70 are located substantially at the mid-point of the arm 10 of the light assembly 4. The light controls 70 allow the light source 30 to be switched on and off and allow the intensity and the colour rendition of the light source 30 to be adjusted. The sterile drape 40 comprises an elongate flexible sock 42 and a substantially transparent portion 50 at the foot of the sock. The flexible sock 42 is made of polypropylene. The substantially transparent portion 50 is optically clear and made of polycarbonate.

The sterile drape 40 is supplied in a sterile pack. Before the lighting system 2 is used, the sterile drape 40 is mounted on the light assembly 4 so that light from the light source 30 exits the lighting system 2 through the substantially transparent portion 50. The drape 40 is retained on the arm 10 by a flexible tie 44 which extends from the drape 40. The tie 44 is wrapped around the outside of the drape 40 and fastened. The tie 44 is fastened so that the drape 40 is constricted around the arm 10.

During use in a medical procedure, an operator 60 can position the light head 20 in any desired position and adjust the light source 30 via the light controls 70 by touching only the sterile drape 40 and not the light assembly 4 itself. In this way, the risk of spreading infection to or from a patient is reduced.

After the procedure, the drape 40 is disposed of and replaced with a new sterile drape.

FIG. 2 shows a lighting system 102 which comprises generally a light assembly 104 and a sterile drape 140.

The light assembly 104 comprises a moveable arm 110 which is (for example) mountable to a stand via a mounting point 112 at a first (proximal) end of the arm 110. The second (distal) end of the arm 110 is connected to a light head 120 by a light head connector 114. The light head connector 114 allows the light head 120 to rotate relative to the arm 110. The arm 110 comprises a first rigid section 113a and a second rigid section 113b which are joined by a rotating joint 111 with a friction lock or spring balancing mechanism. A further rotating joint 111 joins the second rigid section 113b to the mounting point 112. A flexible section 115 connects the first rigid section 113a to the light head connector 114.

An LED light source 130 is mounted in the light head 120. Light controls 170 are located on the light head 120. The light controls 170 allow the light source 130 to be switched on and off and allow the intensity and the colour rendition of the light source 130 to be adjusted.

The sterile drape 140 comprises an elongate flexible sock 142 and a substantially transparent portion 150 at the foot of the sock. The flexible sock 142 and substantially transparent portion 150 are both made from polypropylene.

The sterile drape 140 is supplied in a sterile pack. Before the lighting system 102 is used, the sterile drape 140 is mounted on the light assembly 104 so that light from the light source 130 exits the lighting system 102 through the substantially transparent portion 150. The drape 140 is retained on the arm 110 by the flexible sock 142.

During use in a medical procedure, an operator can position the light head 120 in any desired position and adjust the light source 130 via the light controls 170 by touching only the sterile drape 140 and not the light assembly 104 itself. In this way, the risk of spreading infection to or from a patient is reduced.

After the procedure, the drape 140 is disposed of and replaced with a new sterile drape. FIG. 3 shows a lighting system 202 which comprises generally a light assembly 204 and a sterile drape 240.

The light assembly 204 comprises a moveable arm 210 which is mounted on a bed 300 via a mounting point (not shown) at a first (proximal) end of the arm 210. The second (distal) end of the arm 210 is connected to a light head 220 by a light head connector 214. The light head connector 214 allows the light head 220 to rotate relative to the arm 210.

An LED light source 230 is mounted in the light head 220. Light controls 270 are located on the light head 220. The light controls 270 allow the light source 230 to be switched on and off and allow the intensity and the colour rendition of the light source 230 to be adjusted.

The sterile drape 240 comprises an elongate flexible sock 242 and a substantially transparent portion 250 at the foot of the sock. The flexible sock 242 and substantially transparent portion 250 are both made from polypropylene.

The sterile drape 240 is supplied in a sterile pack. Before the lighting system 202 is used, the sterile drape 240 is mounted on the light assembly 204 so that light from the light source 230 exits the lighting system 202 through the substantially transparent portion 250. The drape 240 is retained on the arm 210 by the flexible sock 242.

During use in a medical procedure, an operator can position the light head 220 in any desired position and adjust the light source 230 via the light controls 270 by touching only the sterile drape 240 and not the light assembly 204 itself. In this way, the risk of spreading infection to or from a patient is reduced.

After the procedure, the drape 240 is disposed of and replaced with a new sterile drape.

### Comparative Example

As a comparative Example, a light assembly with a 50 W tungsten halogen light source (not shown) was tested with a drape similar to drape 240. Within 30 minutes, parts of the drape had melted and the light source had overheated due to the high heat emittance of the 50 W tungsten halogen light source causing excess heat to build up inside the drape. The test was stopped at this point.

A tungsten halogen light source generates substantial amounts of heat in the form of direct infra red radiation from the lamp filament and secondary longer wave infra red radiation from the body of the light head as it gets hot and dissipates heat. The drape trapped this heat causing the temperature of parts of the light assembly to increase to an unacceptable level. This test demonstrates that a standard 50 Watt tungsten halogen examination light operating within a drape may cause the temperature within the drape to rise above the acceptable limits for the materials used in the light assembly and in excess of the safe limits specified in IEC60601-1. AMENDMENTS TO THE CLAIMS

1. A lighting system for medical use comprising:
a light assembly which comprises:
an elongate arm which is surface mountable from a proximal end of the arm;
a light head connected to a distal end of the arm; and
a light source mounted in the light head; and
a drape which comprises:
an elongate flexible body; and
a substantially transparent portion;
wherein the drape is mounted or mountable on and substantially encloses the light assembly such that in use an amount of the light from the light source exits the substantially transparent portion.

2-15. (canceled)

16. The lighting system of claim 1, wherein the elongate flexible body is a sock.

17. The lighting system of claim 16, wherein the substantially transparent portion is located at the foot of the sock.

18. The lighting system of claim 1, wherein the elongate flexible body is open-ended.

19. The lighting system of claim 1, wherein the substantially transparent portion is rigid.

20. The lighting system of claim 1, wherein the substantially transparent portion is optically clear.

21. The lighting system of claim 1, wherein the elongate flexible body comprises a first polymeric material and the substantially transparent portion comprises a second polymeric material.

22. The lighting system of claim 1, wherein the drape is tethered or tetherable to the light assembly.

23. The lighting system of claim 1, wherein the drape is a sterile drape.

24. The lighting system of claim 1, wherein the light source does not emit a significant amount of infra-red radiation.

25. The lighting system of claim 1, wherein the light source has a power consumption of less than 50 W.

26. The lighting system of claim 1, wherein the light source comprises one or more of an LED, a fluorescent light, a gas discharge light or combinations thereof.

27. The lighting system of claim 1, wherein the light source comprises one or more LEDs.

28. A kit of parts comprising:
a light assembly which comprises an elongate arm which is surface mountable from a proximal end of the elongate arm, a light head connected to a distal end of the elongate arm, and a light source mounted in the light head; and
a drape which comprises an elongate flexible body, and a substantially transparent portion;
wherein the drape is mountable on the light assembly such that, in use, the drape substantially encloses the light assembly, and an amount of the light from the light source exits the substantially transparent portion.

29. The kit of parts of claim 28, wherein the elongate flexible body is a sock and the substantially transparent portion is located at the foot of the sock.

30. The kit of parts of claim 28, wherein the elongate flexible body comprises a first polymeric material and the substantially transparent portion comprises a second polymeric material.

31. The kit of parts of claim 28, wherein the light source does not emit a significant amount of infra-red radiation.

32. A sterile drape for a light assembly having an arm which is surface mountable at a proximal end and has a light source at a distal end, the sterile drape comprising:
an elongate flexible body; and
a substantially transparent portion;
wherein the drape is mountable on the light assembly such that, in use, the drape substantially encloses the light assembly, and an amount of the light from the light source exits the substantially transparent portion.

33. The drape of claim 32, wherein the elongate flexible body is a sock and the substantially transparent portion is located at the foot of the sock.

34. The drape of claim 33, wherein the elongate flexible body comprises a first polymeric material and the substantially transparent portion comprises a second polymeric material.
